(19) United States
(12) Patent Application Publication
(10) Pub. No.: US 2017/0224287 A1
(43) Pub. Date: Aug. 10, 2017

(54) PORTABLE APPARATUS FOR MEASUREMENT OF CARDIOVASCULAR HEALTH, SYSTEM, AND METHOD THEREOF

(71) Applicant: KENTEC INC., TAOYUAN COUNTY (TW)

(72) Inventors: CHIA-CHUN LIN, TAICHUNG CITY (TW); KUO-CHING CHIANG, TAOYUAN CITY (TW)

(21) Appl. No.: 15/015,175

(22) Filed: Feb. 4, 2016

(51) Int. Cl. A61B 5/00; A61B 5/021; A61B 5/02; A61B 5/0402

(52) U.S. Cl. CPC A61B 5/7278; A61B 5/0402; A61B 5/02125; A61B 5/02007; A61B 5/0022; A61B 5/0245

(57) ABSTRACT

The disclosure is related to a portable apparatus, and a system for measurement of cardiovascular health, and a method for the same. The portable apparatus is preferably a cuff-less blood pressure device that includes an electrode-based module and a piezoelectric sensor module. The electrode-based module has two electrodes that are used to contact skin of human for generating a first set of data by measuring a target heartbeat. The piezoelectric sensor module is used to generate a second set of data by measuring a pulse wave produced by the target heartbeat corresponding to the first set of data. When the two sets of data are received by a processor, it is able to calculate cardiovascular health value in accordance with cardiovascular health marker that is selected from arterial stiffness, blood pressure, heart rate, pulse transit time, and pulse wave velocity.

### BACKGROUND

1. Technical Field

The present invention is related to an apparatus, a system, and a method for measurement of cardiovascular health in the field of diagnosis, in particular to the apparatus, system and method for identifying the cardiovascular health by monitoring both the electrocardiographic source and piezoelectric pulse source.

2. Description of Related Art

Cardiovascular disease is the leading threat to human health in the world and also a major global health problem. It encompasses a wide gamut of disorders involving the heart and blood vessels that are typically linked. In the developed world, cardiovascular conditions such as stroke and myocardial infarction are leading killers directly caused by atherosclerosis, arterial stiffness, and hypertension.

As with any disease, prevention is far and away the better choice than post-crisis treatment, whenever it is possible. Annually, billions of dollars around the world could be saved in healthcare costs if cardiovascular conditions are well managed before they become life-threatening. Standards of living, particularly for the elderly, would also be dramatically improved if the cardiovascular conditions are caught and prevented at an early stage. To prevent the cardiovascular disease, the blood pressure (BP) is a key issue for the traditional diagnosis and monitoring methods.

The conventional non-invasive blood pressure monitoring method is such as involving a cuff sphygmomanometer that includes an inflatable cuff used to restrict blood flow, e.g. over the arm, and a measuring unit used to determine if the pressure blood flow starts and is unimpeded. However, the traditional cuff sphygmomanometer may be uncomfortable as it is worn on the arm; further, the cuff sphygmomanometer may merely generate the data of the position it is worn and just at the time of use. Therefore, the traditional cuff sphygmomanometer is unable to bear significant medical diagnosis. It can also be inaccurate for various reasons, such as emotional state at the time of measurement, measurement time per day, and the user's mistake of judgment.

Invasive means for blood pressure monitoring, typically such as an intra-arterial catheter, may derive more accurate data and be able to gain constant monitoring of blood pressure. However, the invasive means may run serious risks of arterial injury and infection.

Many modern devices and techniques for monitoring cardiovascular health in the prior art focus on the fixed BP values. Also, the blood pressure in and of itself fails to provide a complete measurement of a particular individual's health since the values of blood pressure and health effects vary greatly between individuals. It is also difficult to achieve the precise fixed systolic/diastolic values by means of BP measuring devices known in the art. These devices tend to provide estimates only, and are non-continuous, momentary spot measurements that are subject to the effects of the individual's emotional and environmental circumstances at the time the measurement is taken.

Algorithms for estimating blood pressure using pulse transit time (PTT) are known in the art, such as Fung et al. “Continuous Noninvasive Blood Pressure Measurement by Pulse Transit Time” Proceedings of the 26th Annual International Conference of the IEEE EMBS, San Francisco, Calif., USA, Sep. 1-5, 2004. Many doctors would agree that a more accurate indicator than a fixed BP value for any particular individual's cardiovascular health is to measure the arterial stiffness and the elasticity of an individual's arterial walls.

The arterial stiffness has been previously measured using both invasive and non-invasive methods. The non-invasive methods tend to fit in three types: 1) measuring Pulse Wave Velocity (PWV), such as with Doppler ultrasound, applanation tonometry, or MRI; 2) relating change in diameter (or area) of an artery to distending pressure, such as with ultrasound or MRI; or 3) assessing arterial pressure waveforms, such as with applanation tonometry. Such methods and comparisons among them can be found in Oliver, James J. and Webb, David J. “Noninvasive Assessment of Arterial Stiffness and Risk of Atherosclerotic Events” Arteriosclerosis, Thrombosis, and Vascular Biology: Journal of the American Heart Association, 2003; 23: 554-566.

Both pulse transit time (PTT) and pulse wave velocity (PWV) have been suggested for assessment of arterial stiffness, such as in Boutouyrie, Pierre et al. “Obtaining arterial stiffness indices from simple arm cuff measurements: the holy grail?” Journal of Hypertension 2009, 27:2159-2161 and Kounalakis, S N and Geladas, N D “The role of pulse transit time as an index of arterial stiffness during exercise” Cardiovasc Eng. 2009 September; 9(3):92-7 Epub 2009 Aug. 6. Health monitoring via smailphones and other mobile devices is a burgeoning industry, with commercially available apps and hardware such as those by iHealth Lab, Inc. and Azumio, Inc. AliveCor has also produced a smailphone ECG which incorporates electrodes into a wireless case that snaps onto the back of a smailphone. Bluetooth heart rate detecting chest straps that communicate with smailphones are known in the art as well, and are commercially available. Examples include those available by Kyto Electronic Co., Ltd. and Dayton Industrial Co., Ltd. Numerous devices or systems that utilize ECG and PPG to measure BP, PTT or other cardiovascular indicators are also known in the art.

### SUMMARY

In the disclosure of the present invention, a portable apparatus for measurement of cardiovascular health, a system, and a method for the same are provided. The system constantly monitors cardiovascular health using an electrocardiography (ECG) source synchronized with a piezoelectric pulse source without requiring invasive techniques, ongoing expensive or large-scale external scanning procedures. The components of the apparatus in accordance with the present invention can collect synchronized measurements on a continuous basis over an extended period of time so as to determine trending of an individual's personal cardiovascular markers over time. The monitoring over time allows for sustained biometric measurements, leading to clarification of an individual user's biometric signature, from which abnormalities in the rate of circulatory degeneration can be determined and the allowing for the application of preventive measures before a health crisis occurs. The invention also provides a method using a mobile device to monitor, either continuously or intermittently, the marker selected from arterial stiffness, blood pressure, heart rate, pulse transit time, and pulse wave velocity.

A portable apparatus, exemplarily the cuff-less blood pressure device, for measurement of cardiovascular health is provided in the present invention. The portable apparatus includes an electrode-based module, being an electrocardiographic source, having at least two electrodes which contact the skin for generating a first set of data by measuring a target heartbeat; a piezoelectric sensor module, being a piezoelectric pulse source, used to generate a second set of data by measuring a pulse wave produced by the target heartbeat corresponding to the first set of data; and a processor, electrically connected with the electrode-based module and the piezoelectric sensor module, in communication with both the electrode-based module and the piezoelectric sensor module for receiving the first set of data and the second set of data and accordingly calculating at least one cardiovascular health value.

In the method for measurement of cardiovascular health by monitoring at least one cardiovascular health marker over time, an ECG signal for a target heartbeat using an electrode-based module of a portable apparatus is obtained and is used to generate a first set of data; after that a piezoelectric pulse signal for a pulse wave from the target heartbeat using a piezoelectric sensor module is also obtained and is used to generate a second set of data. The first set of data and the second set of data are transmitted to the processor for calculating at least one cardiovascular health value in accordance with the at least one cardiovascular health marker using the first and second sets of data.

It is noted that the cardiovascular health marker is selected from arterial stiffness, blood pressure, heart rate, pulse transit time, and pulse wave velocity. The electrode-based module includes at least two electrode contact windows disposed over a housing surface of the portable apparatus and the ECG signal is obtained through the at least two electrode contact windows to contact skin of human body.

In one aspect of the present invention, a system for measurement of cardiovascular health is provided. The system includes one or more portable apparatuses, and a cloud server connected with the one or more portable apparatuses over a network. The cloud server is used to collect the cardiovascular health value from every portable apparatus.

In order to further understand the techniques, means and effects of the present disclosure, the following detailed descriptions and appended drawings are hereby referred to, such that, and through which, the purposes, features and aspects of the present disclosure can be thoroughly and concretely appreciated; however, the appended drawings are merely provided for reference and illustration, without any intention to be used for limiting the present disclosure.

### BRIEF DESCRIPTION OF THE DRAWINGS

FIG. 1 shows a circumstance of a user holding a portable apparatus in their hand in accordance with the present invention;

FIG. 2A and FIG. 2B show the schematic diagrams respectively describing the front panel and back side of the portable apparatus in one embodiment of the present invention;

FIG. 3 shows a block diagram describing the portable apparatus in one embodiment of the present invention;

FIG. 4 shows the diagram depicting the pulse signals for the ECG signals and the piezoelectric pulses in the target heartbeat cycle;

FIG. 5 shows a flow chart describing the method of measurement for cardiovascular health according to one embodiment of the present invention.

### DESCRIPTION OF THE EXEMPLARY EMBODIMENTS

Reference will now be made in detail to the exemplary embodiments of the present disclosure, examples of which are illustrated in the accompanying drawings. Wherever possible, the same reference numbers are used in the drawings and the description to refer to the same or like parts.

Disclosure herein is related to a device for measurement of cardiovascular health, a system and a method for the same. The device is preferably a handheld portable apparatus to conduct the measurement of cardiovascular health. The device is directed to a portable apparatus, applied to a system for measurement of cardiovascular health for identifying the cardiovascular health by monitoring both an electrocardiographic source and piezoelectric pulse source.

For the present invention, acronyms and definitions common in the fields of cardiovascular health and mechanical engineering are to apply. Specific terms include the following:

Electrocardiography (ECG) denotes the measurement of the electrical activity of the heart. An electrocardiogram is the record of the electrical activity of the heart while the signals are detected by electrodes attached to or contacting the skin.

Pulse Wave Velocity (PWV) denotes the velocity at which a pulse wave travels through the arterial tree.

Pulse Transit Time (PTT or T) denotes a time period it takes for a pulse wave to travel between two sites in the arterial tree.

Blood Pressure (BP or P) denotes the pressure exerted by circulating blood upon the walls of blood vessels. In every heartbeat, the blood pressure ranges between a maximum systolic pressure when the heart contracts, and a minimum diastolic pressure when the heart is at rest.

Arterial Stiffness denotes the stiffness of the arterial walls.

BTLE or BLE stands for Bluetooth low energy that represents a feature of Bluetooth 4.2 wireless radio technology.

LCD stands for a liquid crystal display, and is a video display that uses the light modulating properties of liquid crystals.

USB stands for a Universal Serial Bus, and is an industry standard for cables, connectors, and communications protocols between computers and electronic devices.

ADC stands for an Analog-to-Digital Converter, and is a component that uses sampling to convert an analog quantity to a discrete time representation in digital form, i.e. it converts an analog signal into a digital signal.

App stands for application software that is computer software designed to help a user perform a specific task or tasks.

In one embodiment of the present invention, this portable apparatus is such as a cuff-less blood pressure device that can be used to continuously monitor cardiovascular health. The cardiovascular health is monitored while using an electrocardiography (ECG) source synchronized to a piezoelectric pulse source. It is characterized in that the approach is without requiring invasive techniques or ongoing, large-scale external scanning procedures. The portable apparatus includes an electrode-based module acting as an ECG signal source with electrodes contacting the skin for generating a first set of data by measuring a target heartbeat. The portable apparatus includes a piezoelectric sensor module which acts as a PPG (Photoplethysmography) signal source that generates a second set of data by measuring the pulse wave produced by the target heartbeat corresponding to the first set of data. The portable apparatus includes a processor, e.g. a microcontroller or microprocessor, which is configured to receive and process the first set of data and the second set of data. The processor receives the first and second sets of data from which the time differential of the heartbeat pulmonary pressure wave can be calculated. The continuous data related to cardiovascular health markers such as arterial stiffness can be determined.

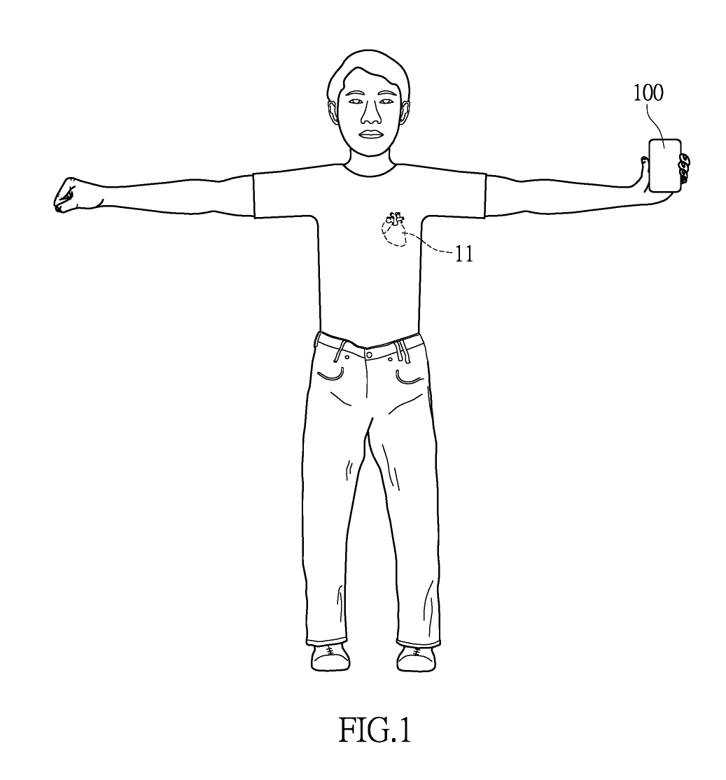

Reference is made to FIG. 1 illustrating a circumstance that a user holds a portable apparatus (100) in their hand to measure their cardiovascular health value. The portable apparatus (100) is such as a cuff-less blood pressure device that includes ECG electrodes and a piezoelectric pulse sensor.

To measure the cardiovascular health value, the user needs to hold the portable apparatus (100) in their hand and at a distance from their heart (11). The portable apparatus (100) is capable of measuring ECG signals using its electrode-based module. The electrode-based module includes at least two electrodes which contact the skin of the user for generating a first set of data by measuring a target heartbeat (11). The portable apparatus (100) is also capable of measuring a pulse wave produced by the target heartbeat (11) using its piezoelectric sensor module. This piezoelectric sensor module is used to generate a second set of data by measuring the pulse wave corresponding to the first set of data.

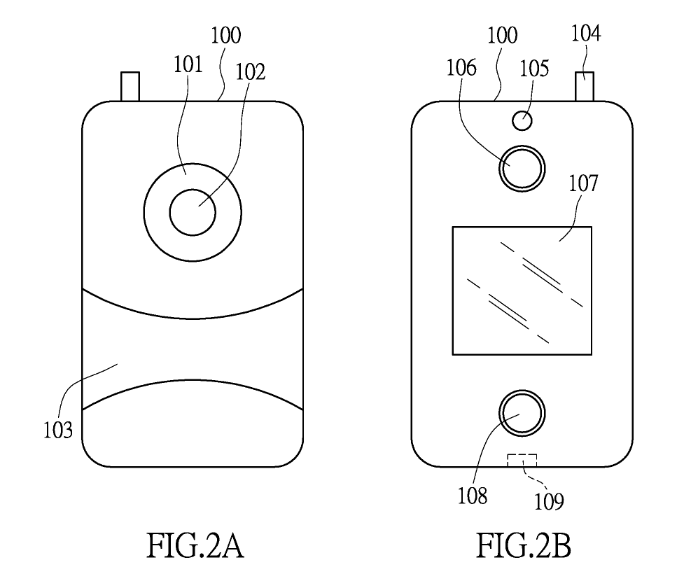

According to one of the embodiments of the present invention, the portable apparatus mentioned in FIG. 1 can be implemented by the schematic diagram described in FIG. 2A and FIG. 2B.

FIG. 2A shows a schematic diagram exemplarily depicting the front panel of the portable apparatus (100). On the front panel, the portable apparatus (100) has a pair of ECG electrodes (101, 103), and a piezoelectric pulse sensor (102). For example, an electrode-based module is included in the portable apparatus (100), and at least two electrode contact windows disposed over a housing surface of the portable apparatus are provided. It is noted that the ECG signal is obtained through the at least two electrode contact windows that contact the skin of the human body.

FIG. 2B schematically shows the back side of the portable apparatus 100. It schematically shows an antenna (104) indicative of its wireless communication capability, e.g. a built-in wireless communication module in compliance with Bluetooth technology. On the back side, the portable apparatus (100) includes an indicator (105) which can be an LED light for indicating the operating status of the apparatus (100). A start key (106) can be used to trigger the function of starting measuring the ECG signals and/or the pulse wave produced by the target heartbeat. Further, an LCD display may be provided for displaying the data produced by the measurement. The LCD display may also show any information coming out of the apparatus (100). A power-on key (108) may be provided on the back side for the user to click to turn on the device. The portable apparatus (100) may be connected to the outside power supply via any communication port such as a USB port (109). Via this USB port (109), the portable apparatus (100) can also communicate with an external device, for example for data synchronization, firmware updating, or a network connection.

It is noted that ECG can be used to measure the rate and rhythm of heartbeat, and this is a measurement of the distance between two consecutive heartbeats. To measure the ECG signals, the user can hold the portable apparatus (100) in one hand, e.g. the left hand shown in FIG. 1, and the skin can contact one of the electrode (103); and then to contact the other hand, e.g. the other electrode (101) contacts around the hand pulse area of the right hand

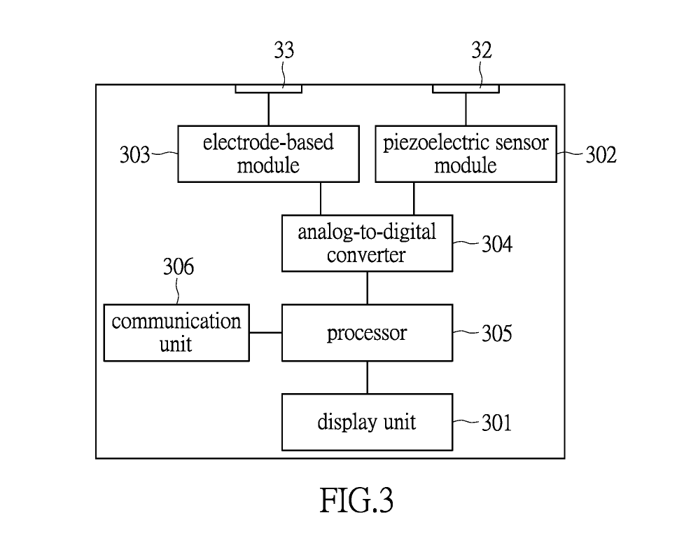

Reference is next made to FIG. 3 depicting a block diagram of the portable apparatus. The block diagram shows several major hardware or software-implemented components of the portable apparatus (100) for embodying the functions of measurement of cardiovascular health according to one embodiment of the present invention.

The internal hardware components of the portable apparatus (100) are such as an electrode-based module (303), a piezoelectric sensor module (302), an analog-to-digital converter (304), a processor (305), and a display unit (301). Further, the portable apparatus (100) may also include a communication unit (306).

In one aspect of the present invention, the processor (305) is such as a microprocessor or a microcontroller adapted in the apparatus (100) for processing the internal operations. The processor (305) is electrically connected with the electrode-based module (303) and the piezoelectric sensor module (302). The processor (305) is essentially in communication with both the electrode-based module (303) and the piezoelectric sensor module (302) for receiving the first set of data and the second set of data and accordingly calculating at least one cardiovascular health value. In one embodiment, the analog-to-digital converter (304) may be used in the circuitry for converting the analog signals obtained via the interfaces (32, 33) to the digital signals for the processor 305.

More, the electrode-based module (303) includes at least two electrodes schematically represented by an electrode interface (33), e.g. a pair of electrode windows, which contact the skin of the human body for generating a first set of data by measuring a target heartbeat. The piezoelectric sensor module (302) may also have a piezoelectric sensor interface (32) used to generate a second set of data by measuring a pulse wave produced by the target heartbeat corresponding to the first set of data.

In one aspect of the present invention, the electrode-based module (303) is an electrocardiographic source that includes at least two electrode contact windows represented by the electrode interface (33) disposed over a surface of the portable apparatus (100). In operation of the portable apparatus (100), the portable apparatus (100) receives analog ECG signals from the electrode-based module (303) via the at least two electrode contact windows (the electrode interface 33) contacted with the skin.

The piezoelectric sensor module (302) may be a piezoelectric pulse source that has the external piezoelectric sensor interface (32). To gain the pulse wave of the heartbeat, the portable apparatus (100) receives the pulse wave from the piezoelectric sensor module (302) via the interface (32).

The analog-to-digital converter (304) is electrically connected with the electrode-based module (303) and the piezoelectric sensor module (302) and is used to convert the analog signals into digital signals that are conveyed to the processor (305).

In one further aspect of the present invention, the portable apparatus (100) includes a communication unit (306). The communication unit (306) may be a wireless communication component for implementing the wireless connection with an external device, or a cloud server for collecting data from the portable apparatus (100).

Furthermore, a pulse transit time (PTT) is one of the cardiovascular health markers for assessing cardiovascular health. To obtain the pulse transit time, the portable apparatus (100) first receives the ECG signals and the piezoelectric pulse from the target heartbeat, and these two sets of data are synchronized by the processor (305) and used to calculate the pulse transit time. The related cardiovascular markers can be therefore derived.

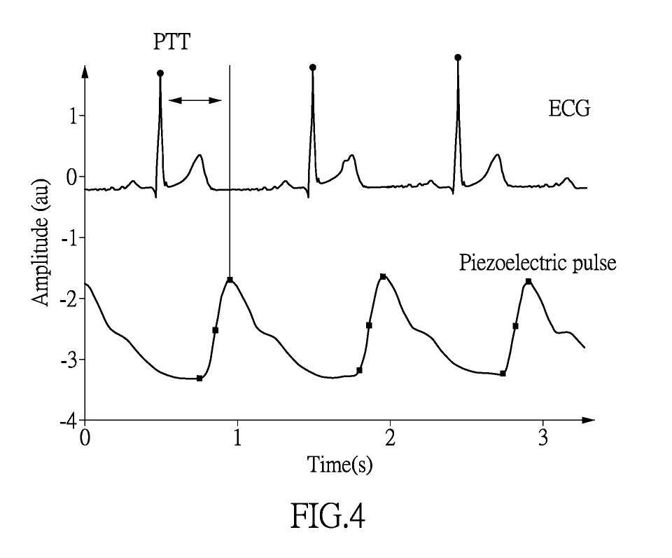

Reference is made to FIG. 4, showing the pulse signals for the ECG signals and the piezoelectric pulses in the target heartbeat cycle.

In the diagram, the peak amplitude of the target heartbeat is detected by the ECG The peak amplitude of the corresponding pulse wave in the blood vessels is detected by the piezoelectric pulse sensor. The difference in time between these peak amplitudes can be recognized as the pulse transit time (PTT). By this method, other cardiovascular health markers can also be calculated. For example, the pulse wave velocity (PWV) is the distance traveling by the pulse divided by PTT (PWV=distance/PTT). It is noted that it is closely approximated by the distance between the ECG and piezoelectric pulse sources, or the measured length of the artery from the target heart to the end of the arm, e.g. the fingers, as exemplarily shown in FIG. 1. The arterial stiffness can also be derived from PWV through the Moens-Korteweg equation.

Alternatively, as referenced in Zhang, Qiao “Cuff-Free Blood Pressure Estimation Using Signal Processing Techniques” Thesis: College of Graduate Studies and Research; Biomedical Engineering; University of Saskatchewan; August 2010 and Hey, Stefan et al. “Continuous noninvasive Pulse Transit Time Measurement for Psycho-physiological Stress Monitoring”

University of Karlsruhe, House of Competence, RG hiper.campus; University of Karlsruhe, Institute for Information Processing Technology; Karlsruhe, Germany, other specific endpoints in the pulse waveform as detected by the PPG may be used to determine the PTT. Possibilities include, but are not limited to, the peak, the midpoint, the foot, the point of maximal slope, and the virtual basepoint. It is noted that the virtual base point corresponds to the intersection point between the tangent to the pulse wave at the point of maximal slope and the horizontal line going through the point having the absolute minimum signal. Different endpoints are suggested to have different advantages in measuring and using the PTT value. For example, using the virtual basepoint has been suggested to give a better virtual noise and artifact robustness. Using the point of maximal slope has been suggested to be strongly related to systolic BP (blood pressure).

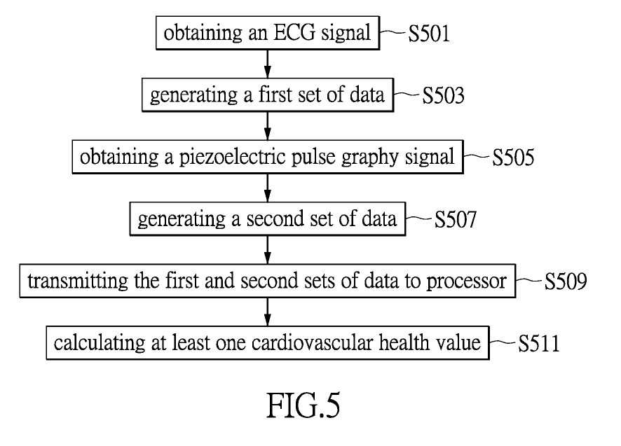

In one further embodiment of the present invention, a method for measurement of cardiovascular health by monitoring at least one cardiovascular health marker over time is disclosed as described in the flow chart shown in FIG. 5.

To obtain the pulse transit time, the embodiments in the disclosure are applicable to the user's handheld portable apparatus that is used to obtain the ECG signals and the piezoelectric pulse signals. The two sets of data are synchronized by the processor of the apparatus and used to calculate pulse transit time and related cardiovascular health markers such as arterial stiffness. In this manner, the method becomes able to continuously derive measurements relating to arterial stiffness and associated cardiovascular markers with this synchronized set of ECG and Piezoelectric Pulse sources. The measurements of data stream sequences are collected from subsequent data ranges and can then be compared to verify cardiovascular trending markers corresponding to a specific individual's rate of arterial stiffness and circulatory degeneration, effectively providing a personalized biometric trending signature. The approach can render preventive measures that can be potentially applied before a health crisis occurs.

In the embodiment of the method shown in FIG. 5, the arterial stiffness of the user who wears the portable apparatus is monitored. The systems including the portable apparatus of the present invention can be configured to measure, calculate, or estimate one or more cardiovascular health markers over time, including, but not limited to, arterial stiffness, blood pressure, heart rate, pulse transit time, and pulse wave velocity. Further, at least two of arterial stiffness, BP, HR, PTT, and PWV can be monitored over time. Still further, at least three of arterial stiffness, BP, HR, PTT, and PWV can be monitored over time. In another embodiment, all of arterial stiffness, BP, HR, PTT, and PWV are monitored over time.

To obtain the data generated by the portable apparatus, a wireless communication technology is used in the portable apparatus. For example, Bluetooth technology may be used to process the communication. More, the portable apparatus may conduct the communication via any data port such as USB. The USB port is one of the schemes that are compatible for computer data transferring and also for battery recharging.

The combination of the signal measurement and data interpolation derived from protracted sequences of continuous monitoring output according to the present invention negates or offsets the need for secondary calibration with an outside source, such as from a cuff. It allows for determination of arterial health trending markers over time. Data derived from a continuous measurement process provides more accurate analysis of cardiovascular health indicators beyond intermittent measurements such as BP, enables derivation of individual biometric trending that can account for anomalous BP, PTT, or PWV values due to moments of stress and other health and environmental triggers.

Continuous, unobtrusive, and portable monitoring approaches disclosed in the present invention can be rendered as strong applications for telemedicine purposes. Without limitation, these monitoring methods are used to remotely validate rehabilitation compliance or fitness goals. It can be used in the doctor's office, in the hospital, at home, and as the individual's daily activities. It has potential for use not only in the medical and fitness fields, but also for monitoring purposes in health insurance, policing, athletics, and military defense. It can be used to remotely store, e.g. via cloud server, or selectively display cardiovascular health data about one or more individuals over a period of time, including during healthy or illness stages, and in determining health marker changes due to disease or aging. Those of ordinary skill in the art could identify a range of practical uses for the present embodiments.

With the sustained synchronous and continuous nature of the present embodiments, there is potential for more accurate PPG measurement of arterial performance efficiency, as opposed to the arterial measurement of the prior art. The portable apparatus, e.g. the cuff-less blood pressure devices, of the present embodiments offers greater usage convenience and portability than the conventional technologies for care of cardiovascular health.

A major advantage of the present invention is the ability to continuously monitor cardiovascular health while involving, at most, two discreet, convenient, portable, unobtrusive devices. An even greater advantage arises with the present invention when the cuff-less blood pressure device comprises both contact ECG electrodes and the Piezoelectric Pulse source, in which all of the components required are in the device to continuously monitor cardiovascular health conveniently and unobtrusively, and those components are housed in a single, compact, handheld device, at a single point on the body.

Links have been suggested throughout the prior art between the values of PTT, PWV, changes in BP, and arterial stiffness. It is generally accepted that both PTT and PWV can be regarded as indices of arterial stiffness, and that both can also be employed as estimators of BP. HR is easily monitored with either the ECG or Piezoelectric Pulse sources by measuring beats per unit time (typically beats per minute, or bpm). The present invention may be configured to calculate or estimate any or all of arterial stiffness, BP, HR, PTT, and PWV through continuous monitoring.

In FIG. 5, a flow chart is shown to disclose the method for measurement of cardiovascular health by monitoring at least one cardiovascular health marker over time in one embodiment of the present invention. Using the portable apparatus essentially including an electrode-based module, a piezoelectric sensor module, and a processor, in step S501, an ECG signal for a target heartbeat can be obtained using the electrode-based module of the portable apparatus. This electrode-based module essentially includes at least two electrodes which are used to contact the skin of the human body, and therefore generates a first set of data by measuring the target heartbeat, such as in step S503. Further, in step S505, a piezoelectric pulse graph signal for a pulse wave from the target heartbeat is obtained using the piezoelectric sensor module of the portable apparatus. The piezoelectric sensor module then generates a second set of data by measuring a pulse wave produced by the target heartbeat corresponding to the first set of data, as shown in step S507.

After that, in step S509, after the first set of data and the second set of data are generated, the data are transmitted to the processor of the portable apparatus through the internal electronic lines. The processor, electrically connected with the electrode-based module and the piezoelectric sensor module, is in communication with both the electrode-based module and the piezoelectric sensor module for receiving the first set of data and the second set of data. In next step S511, by the processor, at least one cardiovascular health value can be calculated in accordance with the at least one cardiovascular health marker using the first and second sets of data.

Furthermore, the system provides a cloud server in one aspect of the present invention, and the cloud server is connected with the one or more portable apparatuses over a network. The cloud server is used to collect the cardiovascular health values from a plurality of portable apparatuses at different locations.

The cardiovascular health marker is selected from arterial stiffness, blood pressure, heart rate, pulse transit time, and pulse wave velocity. For example, in the step for calculating the at least one cardiovascular health marker, the pulse transit time is one of cardiovascular markers and can be calculated at this phase, and can also be continuously monitored. Further, the system can be used to continuously measure arterial stiffness and related cardiovascular markers of the user for the purpose of long-term health monitoring. In a preferable embodiment, the portable apparatus is the major tool to implement long-term monitoring for at least one cardiovascular health marker over time after the at least one cardiovascular health value is calculated.

Accordingly, through prolonged uninterrupted data measurement, the portable apparatus can be used to collect data across multiple independent sequential time ranges and compare it with each other so as to determine a rate of change in accordance with every cardiovascular health marker. Through the system, the rate of change in accordance with every cardiovascular health marker for every portable apparatus can be transmitted to the cloud server. And the cloud server allows every portable apparatus to synchronize the data there-between.

Many prior arts focus on trying to obtain a fixed BP value for an individual to determine their cardiovascular health. Rather than trying to calculate the fixed BP value, one of the objectives of the invention is to determine the degeneration of arterial elasticity over time. In one embodiment, once pulse transit time is calculated, the pulse wave velocity is derived. A suitable formula for linking pulse wave velocity and arterial stiffness is the Moens-Korteweg equation:

PWV=√[(E_{inc}·h)/(2rρ)]

The Moens-Korteweg equation states that PWV is proportional to the square root of the incremental elastic modulus, (E_{inc}), of the vessel wall given constant ratio of wall thickness, h, to vessel radius, r, and blood density, p, assuming that the artery wall is isotropic and experiences isovolumetric change with pulse pressure.

Because of the constant ratio of wall thickness, h, to vessel radius, r, and blood density, ρ, PWV can be used as a direct correlation to arterial stiffness. With monitoring over time, changes in an individual's PWV can be directly linked to changes in arterial stiffness.

Alternately phrased, the Moens-Korteweg equation can be stated as follows:

PWV=√[(tE)/(ρd)]

where t is vessel wall thickness, p is blood density, d is the interior diameter of the vessel. As previously stated, PWV also equals the length of the vessel (L) traveled by the pulse divided by the PTT (T):

PWV=L/T

The elastic modulus, E, is indicated as:

E=E₀e^{αP}

wherein E₀ is the modulus at zero pressure, a is dependent on the vessel, and P is the blood pressure. Making the appropriate combinations and substitutions into the Moens-Korteweg equation yields:

L/T=√[(tE₀e^{αP})/(ρd)]

which leads to:

P=(1/α[ln(L²ρd/tE₀)−(2 ln 7)]

If changes to wall thickness t and diameter of the vessel d with respect to changes to blood pressure P are negligible, and the change in the modulus E₀ is slow enough, the change in blood pressure can be linearly related to the change in PTT as follows:

ΔP=(−2/αT)ΔT

Similarly, through the Bramwell-Hill equation:

PWV=√[(ΔP·V)/(ρ·ΔV)]

where V is blood volume, ΔV is change in blood volume, AP is change in blood pressure, and ρ is blood density.

As found in the prior art through both the Moens-Korteweg and Bramwell-Hill equations, both PWV and PTT have been established to have approximate linear relationships to systolic and diastolic or mean blood pressure (P), according to the following equations:

P=(1/α)(PWV−b), and

P=(1/n)(m−PTT)

where a, b, m, and n are user or patient-specific constants.

The above-mentioned descriptions represent merely the exemplary embodiment of the present disclosure, without any intention to limit the scope of the present disclosure thereto. Various equivalent changes, alterations or modifications based on the claims of the present disclosure are all consequently viewed as being embraced by the scope of the present disclosure.

What is claimed is:

1. A portable apparatus for measurement of cardiovascular health, comprising:
an electrode-based module, comprising at least two electrodes which contact skin for generating a first set of data by measuring a target heartbeat;
a piezoelectric sensor module used to generate a second set of data by measuring a pulse wave produced by the target heartbeat corresponding to the first set of data; and
a processor, electrically connected with the electrode-based module and the piezoelectric sensor module, in communication with both the electrode-based module and the piezoelectric sensor module for receiving the first set of data and the second set of data and accordingly calculating at least one cardiovascular health value.

2. The portable apparatus as recited in claim 1, wherein the apparatus is a cuff-less blood pressure device.

3. The portable apparatus as recited in claim 1, wherein the piezoelectric sensor module is a piezoelectric pulse source.

4. The portable apparatus as recited in claim 1, wherein the electrode-based module is an electrocardiographic source.

5. The portable apparatus as recited in claim 4, wherein the electrocardiographic source comprises at least two electrode contact windows disposed over a surface of the portable apparatus.

6. The portable apparatus as recited in claim 4, further comprising an analog-to-digital converter used to convert the analog signal obtained from the electrode contact windows into a digital signal that is conveyed to the processor.

7. A method for measurement of cardiovascular health by monitoring at least one cardiovascular health marker over time, comprising:
obtaining an ECG signal for a target heartbeat using an electrode-based module of a portable apparatus so as to generate a first set of data;
obtaining a piezoelectric pulse signal for a pulse wave from the target heartbeat using a piezoelectric sensor module of the portable apparatus so as to generate a second set of data;
transmitting the first set of data and the second set of data to a processor of the portable apparatus; and
by the processor, calculating at least one cardiovascular health value in accordance with the at least one cardiovascular health marker using the first and second sets of data.

8. The method as recited in claim 7, wherein the portable apparatus is used to continuously monitor the at least one cardiovascular health marker over time after the at least one cardiovascular health value is calculated.

9. The method as recited in claim 8, wherein the cardiovascular health marker is selected from arterial stiffness, blood pressure, heart rate, pulse transit time, and pulse wave velocity.

10. The method as recited in claim 9, wherein, through prolonged uninterrupted data measurement, further comprising collecting across multiple independent sequential time ranges and comparing with each other so as to determine a rate of change in accordance with every cardiovascular health marker.

11. The method as recited in claim 7, wherein the electrode-based module includes at least two electrode contact windows disposed over a housing surface of the portable apparatus and the ECG signal is obtained through the at least two contact electrode windows to contact skin of human body.

12. A system for measurement of cardiovascular health, comprising:
one or more portable apparatuses, each comprising:
an electrode-based module, comprising at least two electrodes which contact skin for generating a first set of data by measuring a target heartbeat;
a piezoelectric sensor module used to generate a second set of data by measuring a pulse wave produced by the target heartbeat corresponding to the first set of data;
a processor, electrically connected with the electrode-based module and the piezoelectric sensor module, in communication with both the electrode-based module and the piezoelectric sensor module for receiving the first set of data and the second set of data and accordingly calculating at least one cardiovascular health value; and
a cloud server, connected with the one or more portable apparatuses over a network, used to collect the cardiovascular health value from every portable apparatus.

13. The system as recited in claim 12, wherein every portable apparatus continuously monitors the at least one cardiovascular health marker over time for continuously calculating the at least one cardiovascular health value.

14. The system as recited in claim 13, wherein the cardiovascular health marker is selected from arterial stiffness, blood pressure, heart rate, pulse transit time, and pulse wave velocity.

15. The system as recited in claim 12, wherein the electrode-based module of the portable apparatus includes at least two contact electrode windows disposed over a housing surface of the portable apparatus.

16. The system as recited in claim 12, wherein, through prolonged uninterrupted data measurement, the portable apparatus collects across multiple independent sequential time ranges and compares with each other so as to determine a rate of change in accordance with every cardiovascular health marker.

17. The system as recited in claim 16, wherein the rate of change in accordance with every cardiovascular health marker for every portable apparatus is transmitted to the cloud server.

18. The system as recited in claim 17, wherein the cloud server allows every portable apparatus to synchronize the data there-between.
